(19) United States
(12) Patent Application Publication
(10) Pub. No.: US 2012/0328557 A1
(43) Pub. Date: Dec. 27, 2012

(54) ADHESIVE CELL TISSUE GELS

(75) Inventor: Lynn L.H. Huang, Tainan (TW)

(73) Assignees: Excel Med, LLC, Starkville, MS (US); National Cheng Kung University, Tainan (TW)

(21) Appl. No.: 13/167,274

(22) Filed: Jun. 23, 2011

(51) Int. Cl. A61K 38/19; A61K 38/30; A61K 35/12; C08B 37/00; A61K 38/18; C08H 1/00

(52) U.S. Cl. 424/85.1; 514/8.8; 514/9.1; 514/9.2; 514/7.6; 514/8.9; 525/54.1; 424/93.7; 525/54.3; 514/9.6; 514/8.1; 514/9.5; 514/8.4; 514/7.7; 514/8.5

(57) ABSTRACT

Described herein is a cell tissue gel cross-linked with a cross-linking agent, and a quenching agent bound to a reactive group of the cross-linking agent.

### BACKGROUND OF THE INVENTION

Cell tissue gels with adhesive properties have a wide variety of application. Typically, such gels are formed by using cross-linking agents. However, cross-linking agents can be toxic. There is a need for cell tissue gels with reduced toxicity.

### SUMMARY OF THE INVENTION

The present invention is based on an unexpected discovery that a cell tissue gel that is cross-linked with a cross-linking agent and includes a quenching agent capable of binding to a reactive group of the cross-linking agent exhibits improved bounding strength and reduced toxicity.

Accordingly, this invention features a cell tissue gel containing one or more matrix molecules cross-linked with a cross-linking agent, and a quenching agent bound to a reactive group of the cross-linking agent.

The one or more matrix molecules include, but are not limited to, collagen, hyaluronan, gelatin, fibronectin, elastin, tenacin, laminin, vitronectin, heparan sulfate, chondroitin, chondroitin sulfate, keratan, keratan sulfate, dermatan sulfate, carrageenan, heparin, chitin, chitosan, alginate, agarose, agar, cellulose, methyl cellulose, carboxyl methyl cellulose, and glycogen. In some embodiments, the one or more matrix molecules include one of collagen, hyaluronan, and gelatin.

The cross-linking agent typically possesses two bifunctional reactive groups and the bifunctional groups can be identical or different. Cross-linking agents include, but are not limited to, an epoxide, a dialdehyde, a N-hydroxysuccinimide ester, a carbodiimide, genipin, a riboflavin, a flavonoid, a 6-maleimidohexanoic acid active ester, disuccinimidyl suberate, sulfosuccinimidyl-4-(N-maleimidomethyl)cyclohexane-1-carboxylate, and bis(sulfosuccinimidyl)suberate. Quenching agents include, but are not limited to, a diamine, an oligoamine, a polyamine, a dicarboxylic acid, an oligo-carboxylic acid, a polycarboxylic acid, a polysulfhydryl-containing compound, a polyhydroxy-containing compound, and a compound containing heterobifunctional groups.

In some embodiments, the cross-linking agent is genipin and the quenching agent is a poly-L-lysine. The poly-L-lysine can have an average molecular weight of greater than 20 kDa, e.g., greater than 99 kDa, or greater than 212 kDa. In some cases, the cross-linking agent is ethylene glycol diglycidyl ether and the quenching agent is water. When the cross-linking agent is ethylene glycol diglycidyl ether, the quenching can be a poly-lysine or r-polyglutamic acid.

The cell tissue gel of this invention can further contain a nutrient for cell growth (e.g., a cell culture medium), a bioactive agent, and/or cells (e.g., stem cells). The bioactive agent can be a growth factor, e.g., epidermal growth factor, fibroblast growth factor, vascular endothelial growth factor, connective tissue growth factor, platelet-derived growth factor, insulin-like growth factor, nerve growth factor, hepatocyte growth factor, colony-stimulating factor, stem cell factor, keratinocyte growth factor, granulocyte colony-stimulating factor, gramulocyte macrophase colony-stimulating factor, glial derived neurotrophic factor, ciliary neurotrophic factor, endothelial-monocyte activating polypeptide, epithelial neutrophil activating peptide, erythropoietin, bone morphogenetic protein, brain-derived neurotrophic factor, BRAK, transforming growth factor beta, and tumor necrosis factor.

The present invention also features a method of delivering cells (e.g., stem cells) into a subject, including (i) providing a cell implant containing the cell tissue gel described above and cells, and (ii) placing the cell implant in a site of the subject. Also within the scope of this invention is use of the cell tissue gel in manufacturing a cell implant used in cell delivery.

The details of one or more embodiments of the invention are set forth in the description below. Other features or advantages of the present invention will be apparent from the following detailed description of an example and also from the appended claims.

### DETAILED DESCRIPTION OF THE INVENTION

Described herein is a biocompatible cell tissue gel that can support cell growth. The cell tissue gel contains one or more matrix molecules (see below) cross-linked with a cross-linking agent, and a quenching agent bound to a reactive group of the cross-linking agent. The cell tissue gel can also further include a nutrient for cell growth (e.g., a cell culture medium), a bioactive agent, or cells (e.g., stem cells).

Matrix Molecule

A matrix molecule (e.g., a macromolecular compound) helps retain cells at an implantation site. It can be an extracellular molecule found in the extracellular matrix. Examples are, but are not limited to, collagen, hyaluronan, gelatin, fibronectin, elastin, tenacin, laminin, vitronectin, polypeptides, heparan sulfate, chondroitin, chondroitin sulfate, keratan, keratan sulfate, dermatan sulfate, carrageenan, heparin, chitin, chitosan, alginate, agarose, agar, cellulose, methyl cellulose, carboxyl methyl cellulose, glycogen and derivatives thereof. In addition, the matrix molecule can be fibrin, fibrinogen, thrombin, and polyglutamic acid, a synthetic polymer (e.g., acrylate, polylactic acid, polyglycolic acid, or poly(lactic-co-glycolic acid). It is preferred that the matrix molecule used in the tissue gel described herein has a high molecular weight so as to increase the viscosity of the gel.

Any of the naturally-occurring collagens or their functional variants can be used for preparing the tissue gel of this invention. At the present time, at least 28 genetically distinct species of collagens have been discovered. Collagen can be easily isolated and purified from collagen-rich tissues such as skin, tendon, ligament, and bone of humans and animals. Methods for isolating and purifying collagen are well known in the art. (See, e.g., U.S. Pat. No. 5,512,291; U.S. Patent Publication 20040138695; Methods in Enzymology, vol. 82, pp. 33-64, 1982; The Preparation of Highly Purified Insoluble Collagen, Oneson, I., et al., Am. Leather Chemists Assoc., Vol. LXV, pp. 440-450, 1970; U.S. Pat. No. 6,090,996). Collagen can also be prepared by recombinant technology, such as those described by Advanced Tissue Sciences (La Jolla, Calif.) or purchased from various venders (e.g., Fibrogen; South San Francisco, Calif.). One example follows. Bovine deep flexor tendons, with fat and fascia removed, are washed with water, frozen, and sliced into 0.5 mm slices with a slicer. A suitable amount of the sliced tendons is first extracted with 50 ml of water at room temperature for 24 hours. The water-soluble fraction is discarded and the sliced tendons are then extracted with an acidic solution (e.g., 0.2 N HCl) at a suitable temperature (e.g., room temperature) for a suitable period of time (e.g., 12-24 hours). The HCl solution is discarded; the tendons rinsed with water to remove the residual acid. The rinsed tendons are then extracted with a basic solution (e.g., 0.75 M NaOH) at a suitable temperature (e.g., room temperature) for a suitable period of time (e.g., 12-24 hours). After discarding the basic solution, the sliced tendons are neutralized with an acidic solution (e.g., 0.1 N HCl) to a pH of 4-7 (e.g. 5) followed by repetitive washes with water to remove the residual base in the tendons. The tendons are then defatted with an alcohol (e.g., isopropanol) for a sufficient period (e.g., 16 hours) at room temperature. The extractant is decanted and the tendons are further extracted with an alcohol (e.g., isopropanol) for a suitable period (e.g., 12-24 hours) at room temperature to form a collagen-containing solution, which can be dried under a clean hood. The collagen powder thus formed can be dispersed in an acidic lo solution (e.g., 0.5 M or 0.25 M acetic acid) in the presence of a proteolytic enzyme (e.g., trypsin or pepsin) and incubated at 4° C. for a suitable period. The mixture is then filtered through a 100 mesh stainless steel mesh filter and the solubilized collagen can be precipitated with a 5% NaCl solution. The precipitated collagen can be redissolved in the acidic solution described above and the solution thus formed can be filtered through a 100 mesh stainless steel mesh filter to eliminate non-solubilized particles. The collagen solution is then dialyzed with distilled water to remove the acid.

The term “hyaluronan” refers to a naturally-occurring anionic, non-sulfated glycosaminoglycan including repeated disaccharide units of N-acetylglucosamine and D-glucuronic acid, and its derivative. Naturally-occurring hyaluronan (also known as hyaluronic acid or hyaluronate) can be isolated from its natural sources, e.g., capsules of Streptococci, rooster comb, cartilage, synovial joints fluid, umbilical cord, skin tissue and vitreous of eyes, via conventional methods. See, e.g., Guillermo Lago et al. Carbohydrate Polymers 62(4): 321-326, 2005; and Ichika Amagai et al. Fisheries Science 75(3): 805-810, 2009. Alternatively, it can be purchased from a commercial vendor, e.g., Genzyme Corporation, Lifecore Biomedical, LLC and Hyaluron Contract Manufacturing. Derivatives of naturally-occurring hyaluronan include, but are not limited to, hyaluronan esters, adipic dihydrazide -modified hyaluronan, hyaluronan amide products, crosslinked hyaluronic acid, hemiesters of succinic acid or heavy metal salts thereof hyaluronic acid, partial or total esters of hyaluronic acid, sulphated hyaluronic acid, N-sulphated hyaluronic acid, and amines or diamines modified hyaluronic acid. They can be obtained by chemically modifying one or more of its functional groups (e.g., carboxylic acid group, hydroxyl group, reducing end group, N-acetyl group). A carboxyl group can be modified via esterification or reactions mediated by carbodiimid and bishydrazide. Modifications of hydroxyl groups include, but are not limited to, sulfation, esterification, isourea coupling, cyanogen bromide activation, and periodate oxidation. A reducing end group can be modified by reductive amination. It also can be linked to a phospholipid, a dye (e.g., a fluorophore or chromophore), or an agent suitable for preparation of affinity matrices. Derivatives of naturally-occurring hyaluronan can also be obtained by crosslinking, using a crosslinking agent (e.g., bisepoxide, divinylsulfone, biscarbodiimide, small homobifunctional linker, formaldehyde, cyclohexyl isocyanide, and lysine ethyl ester, metal cation, hydrazide, or a mixture thereof) or via internal esterification, photocross-linking, or surface plasma treatment.

It has been shown that hyaluronan, particularly hyaluronan of high molecular weight (i.e., greater than 5 kDa), is effective in promoting angiogenesis, thereby facilitating wound recovery. See U.S. Provisional Application No. 61/390,789. To practice this invention, the hyaluronan can have a molecular weight of 50 kDa to 5,000 kDa (e.g., 70 kDa to 1,500 kDa, 200 kDa to 1,500 kDa, 500 kDa to 1,500 kDa, or 700 kDa to 1,500 kDa).

Also, it has been shown that stem cells display improved viability when growing in a cell tissue gel that contains collagen and hyaluronan at particular weight ratios of 0.01-100 (collagen):1 (hyaluronan). See U.S. application Ser. No. 12/974,535. To make the cell tissue gel described herein, the concentration of the hyaluronan can be 0.001 to 100 mg/ml (e.g., 0.01 to 1 mg/ml) and that of the collagen can be 0.001 to 100 mg/ml. Preferably, the collagen concentration is 0.1 to 100 mg/ml and the hyaluronan concentration is 0.01 to 35 mg/ml. More preferably, the collagen concentration is 3-75 mg/ml (e.g., 6 mg/ml or 9 mg/ml) and the hyaluronan concentration is 0.2-20 mg/ml.

Cross-Linking Agent

A cross-linking agent is capable of reacting with target molecules and links the target molecules together. A cross-linking agent typically contains at least two reactive groups that can react with functional groups of the target molecules. Table 1 below shows exemplary reactive groups and functional groups. Cross-linking agents and reactive groups are well known in the art.

Cross-linking agents useful in the present invention include, but are not limited to, an imidoester, an epoxide (e.g., ethylene glycol diglycidyl ether), a dialdehyde (e.g., glutaraldehyde), a N-hydroxysuccinimide ester (e.g., 2,3-dibromopropionyl-N-hydroxysuccinimide ester, Sulfo-N-hydroxysuccinimide ester, and chlorambucil-N-hydroxysuccinimide ester), a carbodiimide (e.g., 1-ethyl-3-(3-dimethylaminopropyl) carbodiimde hydrochloride), genipin, a maleimide, a haloacetyl, a pyridyl disulfide, a hydrazide, a riboflavin, a bioflavonoid, a flavonoid (e.g., proanthocyanidin, catechin, epicatechin, epigallo catechin, epicatechin gallate, epigallocatechin gallate, quercetin, chalcones, apigenin, luteoiin, a polymethoxylated flavone, quercitoi, kaempferol, myricetin, an anthocyanin, resveritrol, an isoflavanoid, daidzein, genestiein, nobiletin, tangeretin, and tannic acid), a 6-maleimidohexanoic acid active ester, disuccinimidyl suberate, bis(sulfosuccinimidyl)suberate, an azide, a diazirine, sulfosuccinimidyl-4-(N-maleimidomethyl)cyclohexane-1-carboxylate, and derivatives thereof. A cross-linking agent can also be a polymer containing multiple identical or different functional reactive groups, e.g., a polyepoxy compound, and a poly(hydroxy acid).

TABLE 1
Reactive groups and their target functional groups.

| Reactive Group | Target Functional Group |
|---|---|
| Aryl Azide | Nonselective (or primary amine) |
| Carbodiimide | Amine/Carboxyl |
| Carbonyl | Hydrazine |
| Diazirine | Nonselective |
| Hydrazide | Carbohydrate (oxidized) |
| Hydroxymethyl Phosphine | Amine |
| Imidoester | Amine |
| Isocyanate | Hydroxyl (non-aqueous) |
| Maleimide | Sulfhydryl |
| NHS-ester | Amine |
| PFP-ester | Amine |
| Psoralen | Thymine (photoreactive intercalator) |
| Pyridyl Disulfide | Sulfhydryl |
| Vinyl Sulfone | Sulfhydryl, amine, hydroxyl |

Quenching Agent

A quenching agent is an agent capable of reacting with a reactive group of a cross-linking agent. When target molecules are cross-linked with a cross-linking agent, a quenching agent can be used to react with reactive groups of the cross-linking agent that have not reacted with the functional groups of the target molecules. By “using-up” the free reactive groups, a quenching agent can fully or partially reduce the toxicity of the cross-linking agent. A quenching agent can be a compound that contains an amine, a sulfhydryl, a carbonyl, a glycol, a carboxyl, an azide or a photo-crosslinking group. In other words, a quenching agent contains a moiety that can react with a reactive group of a cross-linking agent. For example, for a cross-linking agent with an amine reactive group, the quenching agents include, but are not limited to, diamines, oligoamines, and polyamines such as polylysine and polyglutamine. For a cross-linking agent with a carboxyl reactive group, a useful quenching agent can be a dicarboxylic acid, an oligo-carboxylic acid, or a polycarboxylic acid such as poly-glutamate or poly-glutamic acid. Other exemplary quenching agents include polysulfhydryl-containing compounds, which can be used to quench sulfhydryl reactive groups, and polyhydroxy-containing compounds, which can be used to quench hydroxyl reactive groups.

Nutrient for Cell Growth

The term “nutrient” refers to a source of nourishment essential for cell growth. It can be amino acid, vitamin, mineral, carbon source (e.g., glucose), fatty acid, or a mixture thereof. In one example, the nutrient used in the tissue gel of this invention is a cell growth medium, e.g., Minimum Essential Medium, Basal Medium Eagle, Dulbecco's Modified Eagle's medium, Ham's Nutrient Mixtures F-10 or F-12, Medium 199, RPMI medium, Ames' Media, BGJb Medium (Fitton-Jackson Modification), Click's Medium, CMRL-1066 Medium, Fischer's Medium, Glascow Minimum Essential Medium, Iscove's Modified Dulbecco's Medium, L-15 Medium, McCoy's 5A Modified Medium, NCTC Medium, Swim's S-77 Medium, Waymouth Medium, or William's Medium E.

Bioactive Agent

Any agent (e.g., peptide, polypeptide, oligosaccharide, polysaccharide, or small molecule) that improves cell viability, promotes cell proliferation, or induces cell differentiation can be used in making the tissue gel of this invention. In one example, the bioactive agent is a growth factor, such as epidermal growth factor, fibroblast growth factor, vascular endothelial growth factor, connective tissue growth factor, platelet-derived growth factor, insulin-like growth factor, nerve growth factor, hepatocyte growth factor, colony-stimulating factors, stem cell factor, serotonin, and von Willebrand factor, transforming growth factor, keratinocyte growth factor, granulocyte colony-stimulating factor, granulocyte/macrophage colony stimulating factor, glial derived neurotrophic factor, ciliary neurotrophic factor, endothelial-monocyte activating polypeptide, epithelial neutrophil activating peptide, erythropoietin, bone morphogenetic proteins, brain-derived neurotrophic factor. In another example, the bioactive agent is a cytokine or chemokine, including, but are not limited to, IL-2, breast-expressed chemokine (e.g., BRAK), kidney-expressed chemokine (e.g., CXCL14). The bioactive agent can also be a cell differentiation factor, such as dexamethasone, sodium pyruvate, ascorbic acid-2-phosphate, retinoic acid, proline, insuline, transferrin, selenous acid, linoleic acid, and bovine serum albumin, and TGF-133. In a preferred example, the differentiation factor is a compound that promotes chondrogenesis of mesenchymal stem cells (see those disclosed in U.S. Pat. No. 5,908,784), osteogenesis (e.g., dexamethasone, ascorbic acid, (3-glycerol phosphate), adipogenesis (e.g., insulin, isobutyl-methyl xanthine, dexamethasone, indomethacin), cardiomyogenic differentiation (e.g., activin A, BMP-4), endothelial cell differentiation (e.g., EBM-2, dexamethasone, and VEGF), smooth muscle cell differentiation (e.g., PDGF-BB), neural induction (e.g., bFGF, EGF, and B27 supplement, DMSO, butylated hydroxyanisole, forskolin, valproic acid, KC1, K252a, and N2 supplement) and endodermal lineage differentiation(e.g., dexamethasone, HGF, and FGF-4). The bioactive agent can also be a Chinese herbal medicine or an active ingredient thereof.

Preparation of Tissue Gels with Cell Embedded

The tissue gel described herein can be prepared by mixing all of its components mentioned above at a desired weight ratio and keeping the mixture under suitable conditions to allow gel formation. To prepare a cell-embedded gel, desired cells can be mixed with the gel components prior to gel formation. The cells can be stem cells obtained from a mammal (e.g., bovine, porcine, murine, equine, canine, feline, ovine, simian, and human). Examples are, but are not limited to, placenta-derived stem cells, bone marrow-derived stem cells, stromal cells (e.g., adipose-derived stromal cells), mesenchymal stem cells, tissue progenitor cells, blast cells, or fibroblasts.

The tissue gel thus prepared, with cells embedded, can be implanted to a desired site for tissue repair and other therapeutic purposes.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All references cited herein are incorporated by reference.

### EXAMPLE

### Cross-Linked Tissue Gels Containing Poly-L-Lysine

(A) Materials and Methods

The quenching agents tested in the study include spermine, protamine, 1,6-hexanediamine, poly-L-lysines with different sets of molecular weights. The average molecular weights of the poly-L-lysines were 3.4, 20, 99, 212 and 225 kDa.

Equal normality of each of the polyamines was premixed with gelatin (300 mg/mL) in a phosphate buffered saline solution. Equal volume of a genipin solution at 20 mg/mL and the above polyamine-gelatin solution were mixed before applying to the dermal side of a pig skin sample (10×30×0.7˜0.9 mm³) to form an adhesive. A weight of 50 gm was placed on the area with the adhesive (1×1 cm²) for 30 minutes. The bounding strength of the adhesive was measured using the LRX Materail Testing System (Lloyd Instruments Ltd., England) at a separation rate of 10 mm/min.

The cytotoxic effect of the adhesive was evaluated by applying 10 μL of the adhesive to the center of a well of a 24-well plate. Fibroblasts (2.0×10⁴ cell/well) were seeded and incubated at 37° C. with 5% CO₂ for 38 hours. The number of viable cells was quantified by a hemacytometer.

The rheological properties of the adhesives in the presence and absence of the poly-L-lysine were measured by a set of cone and cup using a RheoStress RS 150 (Haake, Germany). The elastic storage modulus (G′) in real time was recorded along with time at fixed shear stress (1 Pa) and frequency (1 Hz). The effects of reaction temperature on G′ were also monitored.

(B) Bounding Strength

The results demonstrate that polyamines at molecular weight above 20 kDa improved the bounding strength of the adhesives. Poly-L-lysine at molecular weight of 212 kDa was chosen to perform further studies. These further studies showed that the bounding strength of an adhesive with the poly-L-lysine increased along with elevated concentration of poly-L-lysine until 46.8 mN. Studies done with an adhesive made with 17.5 mN poly-L-lysine and one without any polylysine showed that the maximum bounding strength of an adhesive with the polylysine is higher than one without the polylysine.

Elevated bounding strengths of the adhesive were observed with increased amounts of poly-L-lysine regardless of the concentration of genipin. In addition, the bounding strength was enhanced with raised concentration of genipin.

(C) Toxicity

Cytotoxicity of the adhesive in the absence of poly-L-lysine was increased with elevated concentration of genipin. The addition of poly-L-lysine significant reduced the cytotoxic effects of genipin. No significant cytotoxic effect was observed with 7.5 mg/mL of genipin together with 46.8 mN of a poly-L-lysine.

(D) Rheological Properties

To further understand the influence of the poly-L-lysine on the physical and chemical properties of the adhesive, the elastic storage modulus (G′) in real time was monitored. Through the oscillation test with fixed shear stress (1 Pa) and frequency (1 Hz), the G′ value increased along with the formation of the adhesive from a liquid state. The G′ value was significantly higher with the addition of poly-L-lysine to the adhesive. The G′ values at different reaction temperatures were monitored and a steep increase at about 90 minutes was observed with the reaction carried out 50° C. The phase transition indicated that the poly-L-lysine was involved in the cross-linking reaction of the adhesive and greatly increased the elastic modulus of the adhesive. The phase transition was not observed in the absence of the poly-L-lysine.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A cell tissue gel, comprising one or more matrix molecules cross-linked with a cross-linking agent, and a quenching agent bound to a reactive group of the cross-linking agent.

2. The cell tissue gel of claim 1, wherein the cross-linking agent is selected from the group consisting of an epoxide, a dialdehyde, a N-hydroxysuccinimide ester, a carbodiimide, genipin, a riboflavin, a flavonoid, a 6-maleimidohexanoic acid active ester, disuccinimidyl suberate, sulfosuccinimidyl-4-(N-maleimidomethyl)cyclohexane-1-carboxylate, and bis(sulfosuccinimidyl)suberate.

3. The cell tissue gel of claim 1, wherein the quenching agent is selected from the group consisting of a diamine, an oligoamine, a polyamine, a dicarboxylate, an oligo-carboxylate, a polycarboxylate, a polysulfhydryl compound, and a polyhydroxy compound.

4. The cell tissue gel of claim 1, wherein the cross-linking agent is genipin and the quenching agent is a poly-L-lysine.

5. The cell tissue gel of claim 4, wherein the poly-L-lysine has an average molecular weight of greater than 20 kDa.

6. The cell tissue gel of claim 5, wherein the poly-L-lysine have an average molecular weight of 99 kDa or greater.

7. The cell tissue gel of claim 5, wherein the poly-L-lysine has an average molecular weight of 212 kDa or greater.

8. The cell tissue gel of claim 1, wherein the cross-linking agent is ethylene glycol diglycidyl ether and the quenching agent is water.

9. The cell tissue gel of claim 1, wherein the cross-linking agent is ethylene glycol diglycidyl ether and the quenching agent is a poly-lysine or r-polyglutamic acid.

10. The cell tissue gel of claim 1, wherein the one or more matrix molecules are selected from the group consisting of collagen, hyaluronan, gelatin, fibronectin, elastin, tenacin, laminin, vitronectin, heparan sulfate, chondroitin, chondroitin sulfate, keratan, keratan sulfate, dermatan sulfate, carrageenan, heparin, chitin, chitosan, alginate, agarose, agar, cellulose, methyl cellulose, carboxyl methyl cellulose, and glycogen.

11. The cell tissue of claim 10, wherein the one or more matrix molecules are selected from the group consisting of collagen, hyaluronan, and gelatin.

12. The cell tissue gel of claim 11, wherein the one or more matrix molecules are collagen and hyaluronan.

13. The cell tissue gel of claim 4, wherein the one or more matrix molecules include gelatin.

14. The cell tissue gel of claim 8, wherein the one or more matrix molecules include collagen.

15. The cell tissue gel of claim 9, wherein the one or more matrix molecules include gelatin.

16. The cell tissue gel of claim 1, further comprising a nutrient and a bioactive agent.

17. The cell tissue gel of claim 16, wherein the nutrient is a cell culture medium.

18. The cell tissue gel of claim 16, wherein the bioactive agent is a growth factor selected from the group consisting of epidermal growth factor, fibroblast growth factor, vascular endothelial growth factor, connective tissue growth factor, platelet-derived growth factor, insulin-like growth factor, nerve growth factor, hepatocyte growth factor, colony-stimulating factor, stem cell factor, keratinocyte growth factor, granulocyte colony-stimulating factor, gramulocyte macrophase colony-stimulating factor, glial derived neurotrophic factor, ciliary neurotrophic factor, endothelial-monocyte activating polypeptide, epithelial neutrophil activating peptide, erythropoietin, bone morphogenetic protein, brain-derived neurotrophic factor, BRAK, transforming growth factor beta, and tumor necrosis factor.

19. The cell tissue gel of claim 16, further comprising stem cells.

20. The cell tissue gel of claim 12, wherein the collagen and the hyaluronan are at a weight ratio of 0.01-100:1.

21. A method of delivering stem cells into a subject, comprising:
providing a cell implant containing the cell tissue gel of claim 1 and stem cells, and placing the implant in a site of the subject.
